(19) United States
(12) Patent Application Publication
(10) Pub. No.: US 2026/0325774 A1
(43) Pub. Date: Oct. 22, 2026

(54) METHOD AND SYSTEM FOR DETERMINING THE AMOUNT OF OXYGEN REQUIRED BY A USER WITH RESPIRATORY PROBLEMS

(71) Applicants: UNIVERSITAT POLITECNICA DE CATALUNYA, Barcelona (ES); Fundació de Recerca Clínic Barcelona-Institut d’Investigacions Biomèdiques August Pi i Sunyer, Barcelona (ES); HOSPITAL CLÍNIC DE BARCELONA, Barcelona (ES)

(72) Inventors: Xavier MASIP BRUIN, Sant Pere de Ribes (ES); Adrià TORT MARTINEZ, Vilanova i la Geltrú (ES); Albert ALONSO BELTRÁN, Vilanova i la Geltrú (ES); Isabel BLANCO VICH, Barcelona (ES)

(73) Assignees: UNIVERSITAT POLITECNICA DE CATALUNYA, Barcelona (ES); Fundació de Recerca Clínic Barcelona-Institut d’Investigacions Biomèdiques August Pi i Sunyer, Barcelona (ES); HOSPITAL CLÍNIC DE BARCELONA, Barcelona (ES)

(21) Appl. No.: 19/532,669

(22) Filed: Feb. 6, 2026

Related U.S. Application Data
(63) Continuation-in-part of application No. 17/916,521, filed on Sep. 13, 2021, now abandoned.

(30) Foreign Application Priority Data
Oct. 1, 2020 (EP) 20382872.8

(51) Int. Cl. A61M 16/00

(52) U.S. Cl. CPC A61M 16/026; A61M 16/0051; A61M 2202/0007; A61M 2202/0208; A61M 2205/18; A61M 2205/3303; A61M 2205/3306; A61M 2205/3327; A61M 2205/3334; A61M 2205/502; A61M 2205/581; A61M 2205/583; A61M 2205/8206; A61M 2230/005; A61M 2230/06; A61M 2230/20

(57) ABSTRACT

A method for determining the amount of oxygen to be delivered to a user with respiratory problems is disclosed. The method computes a behavioral model of the user using an artificial intelligence algorithm on monitoring data of the user, the latter being collected while the user performs an activity and comprising oxygen saturation data; collects follow-up monitoring data of the user while the user performs an activity using an oxygen supply device, the follow-up monitoring data comprising oxygen saturation data; computes an estimated amount of oxygen to be delivered to the user based on the collected follow-up monitoring data and the behavioral model; and adjusts the behavioral model by training at least one artificial intelligence algorithm that uses the follow-up monitoring data. In other embodiments, instead of computing the behavioral model, the method uses a base behavioral model.

### TECHNICAL FIELD

The present invention relates to a method and system for determining the amount of oxygen required by a user/patient with respiratory problems.

### BACKGROUND OF THE INVENTION

There are known some devices that allow regulating automatically and continuously an amount of oxygen delivered to a patient/user while constantly acquiring physiological parameters of the patients/users, for example the O2 Flow Regulator of the company Dima Italia™ or Sanso Via™ of the company Sanso Health.

Likewise, OXITONE™ provides a SaaS solution focusing on monitoring patients (position, pulse, saturation) and health professionals with the collected data and some statistics through a dashboard.

### DESCRIPTION OF THE INVENTION

An object of the present invention is to provide a highly efficient, dynamic, adaptative, usable and secure solution aimed at monitoring and caring patients/users with chronic respiratory diseases, technically supported by a novel patients’ behavior modelling solution through an Artificial Intelligence (AI) engine and a well-defined caring methodology covering the whole disease lifecycle, i.e., assisting the initial diagnose steps and providing a mechanism to adapt oxygen delivery either alone or associated with ventilatory support devices (BIPAP, CPAP, etc.), intended to support the needs for real-time oxygen supply in said patients/users. Indeed, the present invention focusses on significantly improving patients’ quality of life by increasing their tolerance to the activities of daily living and avoiding sedentary behaviors.

To that end, the present invention provides a method for determining the amount of oxygen to be delivered to a user/patient with respiratory problems.

According to a first aspect, the method comprises computing, by a processing unit, a behavioral model of the patient/user, the behavioral model characterizing a relationship between oxygen saturation and physical effort of the patient/user, wherein computing the behavioral model comprises training at least one artificial intelligence algorithm using monitoring data from the patient/user, the monitoring data being collected by a wearable data acquisition device while the patient/user performs at least one activity and comprising at least oxygen saturation data; collecting, by the wearable data acquisition device, follow-up monitoring data from the patient/user while the user performs an activity, the follow-up monitoring data comprising at least oxygen saturation; computing, by a processing unit, an estimated amount of oxygen to be delivered to the patient/user based on the collected follow-up monitoring data and the behavioral model; and adjusting, by a processing unit, the behavioral model using at least one artificial intelligence algorithm that utilizes the follow-up monitoring data.

According to a second aspect, the method comprises using, by a processing unit, a base (or generic) behavioral model, the base behavioral model being selected from a set of previously computed behavioral models utilizing existing data from different patients/users, based on at least one of a disease type of the user, an effort capacity (or result of the effort capacity) of the patient/user, or an input provided by a healthcare staff member; collecting, by the wearable data acquisition device, follow-up monitoring data from the patient/user while the patient/user performs an activity, the follow-up monitoring data comprising at least oxygen saturation data; computing, by a processing unit, an estimated amount of oxygen to be delivered to the patient/user based on the collected follow-up monitoring data and the base behavioral model; and adjusting, by a processing unit, during a first iteration, the base behavioral model using at least one artificial intelligence algorithm that utilizes the follow-up monitoring data, generating an adjusted behavioral model as a result, and subsequently adjusting the adjusted behavioral model during successive iterations.

Patient/user follow-up to monitor disease progression may be performed at predetermined periods or intervals, for example as prescribed by medical staff responsible for the patient/user’s care. Accordingly, follow-up monitoring data may be acquired from the patient/user on a daily basis, every two days, weekly, or monthly. The patient/user behavioral model is updated and retrained upon each acquisition of follow-up monitoring data, based on the newly collected data, until the behavioral model reaches a predefined accuracy threshold. Upon reaching the accuracy threshold, a new patient/user behavioral model may be generated. In embodiments that employ a base behavioral model, the adjusted base behavioral model is used in subsequent monitoring and training steps generating a new behavioral model. The predefined threshold may be further updated to adapt for example to new requirements and specifications, demanding for additional adjustments in the behavioral model. Various aspects of the proposed method, as described herein, may be embodied in programming. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors, or the like, or associated modules thereof, such as various semiconductor memories, tape drives, disk drives and the like, which may provide storage at any time for the software programming.

In some embodiments, the collection of the follow-up monitoring data may be performed while the patient/user is using an oxygen supply device or concentrator.

In some embodiments, the monitoring data and the follow-up monitoring data further comprise one or more of pulse or heart rate (HR) data, geolocation data, oxygen concentrator flow rate data, predicted oxygen saturation (SpO₂) data, accelerometer data, wearable data acquisition device status information including battery status information, communication status information, and information relating to one or more nearby computing or electronic devices.

The artificial intelligence algorithms considered by the present invention are dynamic, intended to easing the adoption of novel AI solutions and removing those showing low accuracy. In certain embodiments, at least one neural network is employed. In other embodiments, the artificial intelligence algorithms may comprise one or more of supervised learning algorithms, unsupervised learning algorithms, reinforcement learning algorithms, evolutionary and optimization algorithms, and logic-based or knowledge-based systems.

In some embodiments, the calculation of the behavioral model and/or the training of the behavioral model/base behavioral model can further use synthetic data generated by artificial intelligence techniques (e.g. GenAI).

In some embodiments, the activity comprises a physical activity, for example, walking via a known or unknown route, exercises programmed by a physiotherapist, etc., or a daily activity at the monitored patient/user’s home. In some embodiments, the activity comprises the effort test known as the 6 minute walking test.

In an embodiment, the method also displays the patient/user’s behavioral model and/or the estimated amount of oxygen on a screen of a computing device.

In an embodiment, the method also comprises sending an audible and/or visual warning signal to the monitored patient/user and/or to a healthcare staff if the estimated amount of oxygen is different than a threshold. In addition, in the event of detecting an emergency, such as, arrhythmias, signs of cardiac arrest, cardiac arrest, severe desaturation, very low/high blood pressure, etc., the method can further instruct an oxygen delivery element via a user’s electronic device, such as a mobile device, for example, a Smartphone, among others, to apply a fixed minimum dose of oxygen to the patient/user, and alert technical operators, specialists and/or emergency services.

Present invention also provides, according to another aspect, a system for determining the amount of oxygen to be delivered to a patient/user with respiratory problems. The system includes a memory or database, a wearable data acquisition device adapted to be placed on a monitored patient/user having respiratory problems, and one or more processing units operatively associated with or connected to the wearable data acquisition device.

The memory or database is adapted to store monitoring data and follow-up monitoring data of the patient/user acquired/collected via the wearable data acquisition device while the patient/user performs an activity.

In the proposed system, the one or more processing units are configured to: compute a behavioral model characterizing a relationship between oxygen saturation and physical effort of the patient/user or use a base behavioral model; compute an estimated amount of oxygen to be delivered to the patient/user based on the collected follow-up monitoring data and the behavioral model or base behavioral model; and adjust the behavioral model/base behavioral model by training the model using at least one artificial intelligence algorithm executed on the follow-up monitoring data.

The one or more processing units can be included either in any computing device (e.g., a smartphone, microcontroller, etc.) or in any computing server/unit (e.g., a cloud computing server). In some embodiments, one of the processing units is included in a cloud computing server which is configured to perform the computing of the behavioral model, and one of the processing units is included in a smartphone or a computing device (such as an ad-hoc device) which is configured to compute the estimated amount of oxygen.

The wearable data acquisition device comprises one or more sensors, such as an oxygen saturation level sensor and a heart rate sensor. Additionally, the one or more sensor can also include a GPS or geolocation sensor, a pulse (HR) sensor, an accelerometer, a concentrator oxygen flow rate sensor, etc.

In an embodiment, the wearable data acquisition device further includes an oxygen delivery element such as a cylinder valve, oxygen concentrator, portable oxygen concentrator, oxygen outlet, among others.

In an embodiment, the wearable data acquisition device may be located on the wrist, head, forearm, finger, ankle, calf, among others.

Other embodiments of the invention that are disclosed herein also include software programs to perform the method embodiment steps and operations summarized above and disclosed in detail below. More particularly, a computer program product is one embodiment that has a computer-readable medium including computer program instructions encoded thereon that when executed on at least one processor in a computer system causes the processor to perform the operations indicated herein as embodiments of the invention.

Thus, the present invention can provide:
Integrated solution including two well defined phases, planning (modelling patient/user’s behavior) and runtime (estimating real-time patients/users’ oxygen support needs).
Agnostic to the oxygen system provider.
Supporting external data sources for improved decision making.
Heartbeat, saturation monitoring, and other monitoring metrics.
AI Engine estimating patients/users’ performance modeling and oxygen support needs.
Sampling period customized and adaptable.
2 dashboards, Frontend for health professionals (F4H) and Frontend for Live (F4L) for health professionals and patients respectively, both connected through a Backend system and patient database.
Data sensitive care (privacy concerns).
Distributed smartness and data analytics at cloud.
Routes training protocol for exercise pattern recommendation.
Adherence support.
Warnings to both the patient/user and the health professionals’ team.
Diagnose recommendation to the health professionals’ team.
Real-time patient/user monitoring.
Real-time oxygen adaptation to the needs of the patient/user.
Direct connectivity between all involved actors (patient/user, caregivers, professionals).
Warnings to the oxygen provider about real status (capacity, etc.) of its device.
Micro-services oriented architecture.

### BRIEF DESCRIPTION OF THE DRAWINGS

The previous and other advantages and features will be more fully understood from the following detailed description of embodiments, with reference to the attached figures, which must be considered in an illustrative and non-limiting manner, in which:

FIG. 1 schematically illustrates the blocks architecture of the present invention, according to an embodiment.

FIG. 2 schematically illustrates the planning phase, according to an embodiment.

FIG. 3 graphically illustrates an example of how the behavioral model is downloaded in the runtime phase.

FIG. 4 graphically illustrates an example of how the behavioral model is queried and the estimator acquired in the runtime phase.

FIG. 5 shows another embodiment of the functional architecture of the present invention.

FIG. 6 schematically illustrates the backend architecture, according to an embodiment.

FIG. 7 schematically illustrates the dashboard plus routes architecture, showing how the dashboards and route Systems (all optional) are connected to the backend System.

### DETAILED DESCRIPTION OF THE INVENTION

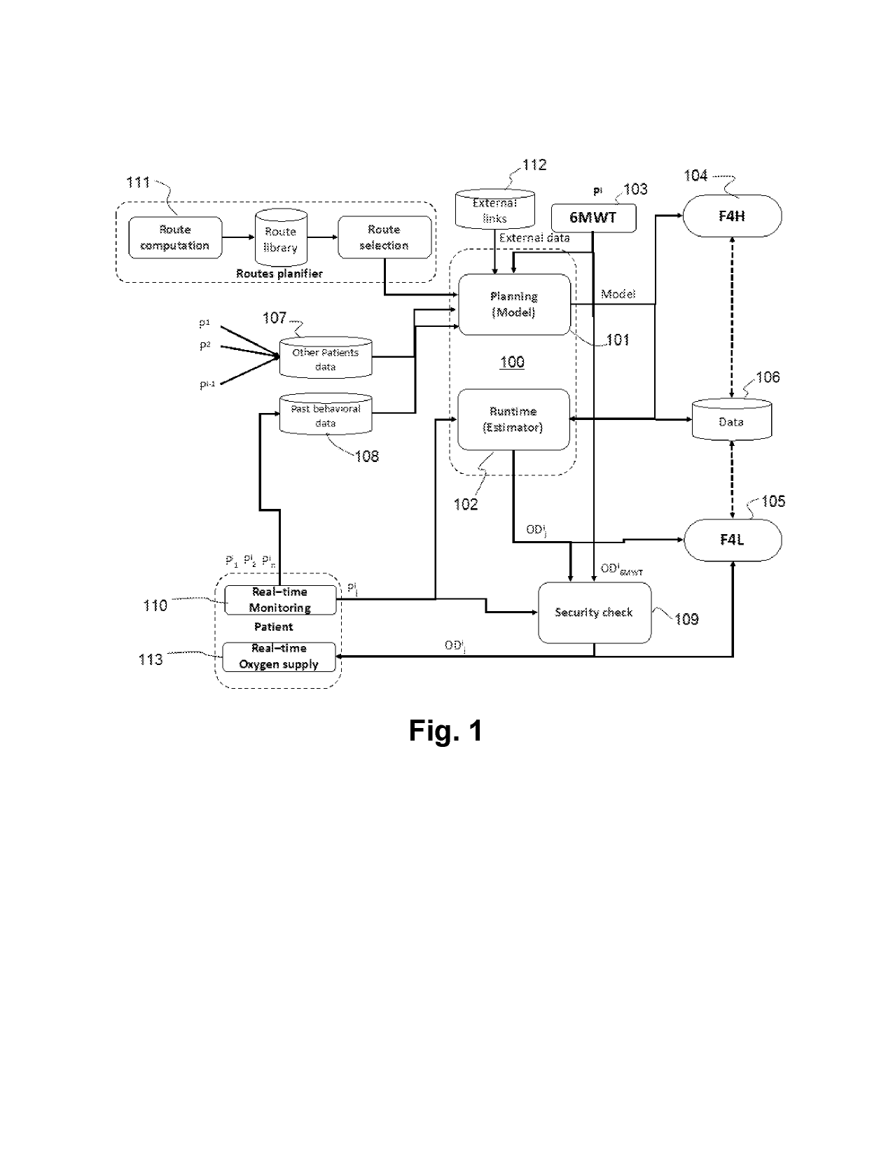

FIG. 1 shows an embodiment of the proposed architecture of the invention representing a two-phase design supported by the AI engine 100. All additional features and functionalities as defined in the present invention are inferred from these two-phase design, including adherence, warnings, communication, routes training, etc.

According to this embodiment, a planning phase 101 is fed by data collected from the 6 minute walking test (6MWT) 103, which is collected at hospital premises in a highly controlled scenario. In some cases, in particular when the individual patients/users 1 only run the 6MWT once per month, or even less, during the planning phase 101 data replication for individual patients/users will not be considered. In fact, according to the current protocol, health professionals estimate the oxygen dose to be delivered to the patients after usually running only once the 6MWT, according to a fixed table mapping oxygen saturation into oxygen delivery. A runtime phase 102 will collect users/patients’ data from their daily activities (walking, etc.), thus putting together a large set of individualized information for each individual user 1. This information is used by the planning phase 101 in next iterations.

With regard to the AI Engine 100, each phase 101, 102 include a block deploying specific AI-based solutions. At the start, the input for the planning phase 101 for each individual patient/user 1 will be the overall set of data collected (a maximum can be set) for patients/users with similar characteristics (disease, age, gender, etc.), i.e. the first data as termed in the claims, along with results of the individual user 6MWT, i.e. the second data as termed in the claims. In further iterations of the proposed method, past patient/user’s activity data (i.e. third data) will be also used by the planning phase 101. In this way a behavioral model is computed for each individual patient/user 1. Indeed, the output of the planning phase 101 is an individual patient/user’s behavioral model, to be daily trained with updated data coming from daily user’s activity monitoring (i.e. collected by the real time monitoring module 110 that is sensing/monitoring the patient/user 1). In an embodiment, the process of updating/adjusting the behavioral model is done daily, usually when there is no user activity and all data for that day is collected (normally at nights).

Consequently, the planning phase outcome is a patient/user’s behavioral model modeling patient’s saturation vs the expected effort, which as days goes on, is continuously trained and updated and so becoming much more accurate.

All collected and processed information is stored in a backend repository and made accessible to health professionals and patients/users through two customized dashboards 104, 105, the F4H and F4L respectively. The two dashboards 104, 105 facilitate patients/users and professionals to get connected to the system on each phase and to easily get the required information.

When the patient/user 1 performs an activity at home or outdoors, such as walking, the runtime phase 102 starts. In this phase 102 the individual patient/user 1 is continuously monitored in previously defined periods of time, for instance every 1-5 minutes, in particular 1 minute, and the collected values are used to query the computed behavioral model turning into an estimator of the oxygen needs for the next period, i.e., the next minute. The data collected in this phase 102 via the cited real time monitoring module 110 updates (particularly in a daily basis) previous data used in the planning block 101 and that is stored in the database 108, thus adjusting the behavioral model accuracy and thus increasing the chances for the runtime block 102 to deliver an oxygen prediction much closer to real patient/user’s needs. The outcome of the AI Engine 100, i.e., the behavioral model and the oxygen estimator, will be better as larger the information collected from the patient/user 1 is. Hence, in some embodiments, to prevent deviations, mainly in the first weeks, a security check 109 is introduced intended to correct sudden large changes in the oxygen estimation. In this case, when a previously threshold is passed, the basal oxygen computed according to the 6MWT, is recommended to the patient/user 1.

Finally, the output of the security check 109 goes to the oxygen-supply module 113 to deliver/provide the estimated amount/quantity of oxygen to the patient/user 1.

As also indicated in FIG. 1, the present invention also facilitates the inclusion of a set of categorized routes (Routes planner 111) to support the exercise recommendation to be given by the health professional to the patient/user 1. To that end, the present invention, in an embodiment, delivers advice at the planning phase 101 and updates the recommendation while monitoring and self-learning about the patient/user behavior.

The smart decision process proposed by the present invention can be distributed throughout cloud and local facilities, and is supported by the cited AI Engine 100, consisting in the AI-assisted strategy technically providing the desired objectives on each phase 101, 102. It must be emphasized that both phases 101, 102 are active, each in different time windows. Indeed, the planning phase 101 remains active when the patient/user 1 is performing no activity (in other words, is not producing data), training the behavioral model. Differently, the runtime phase 102 is active when the patient/user 1 activity is monitored, collecting data sent to the database and querying the computed model to deliver the proper oxygen estimation. To this end, the patient/user 1 should wear a wearable data acquisition device and a software APP running in a mobile device 10 (e.g. a Smartphone) should be open.

### Planning Phase

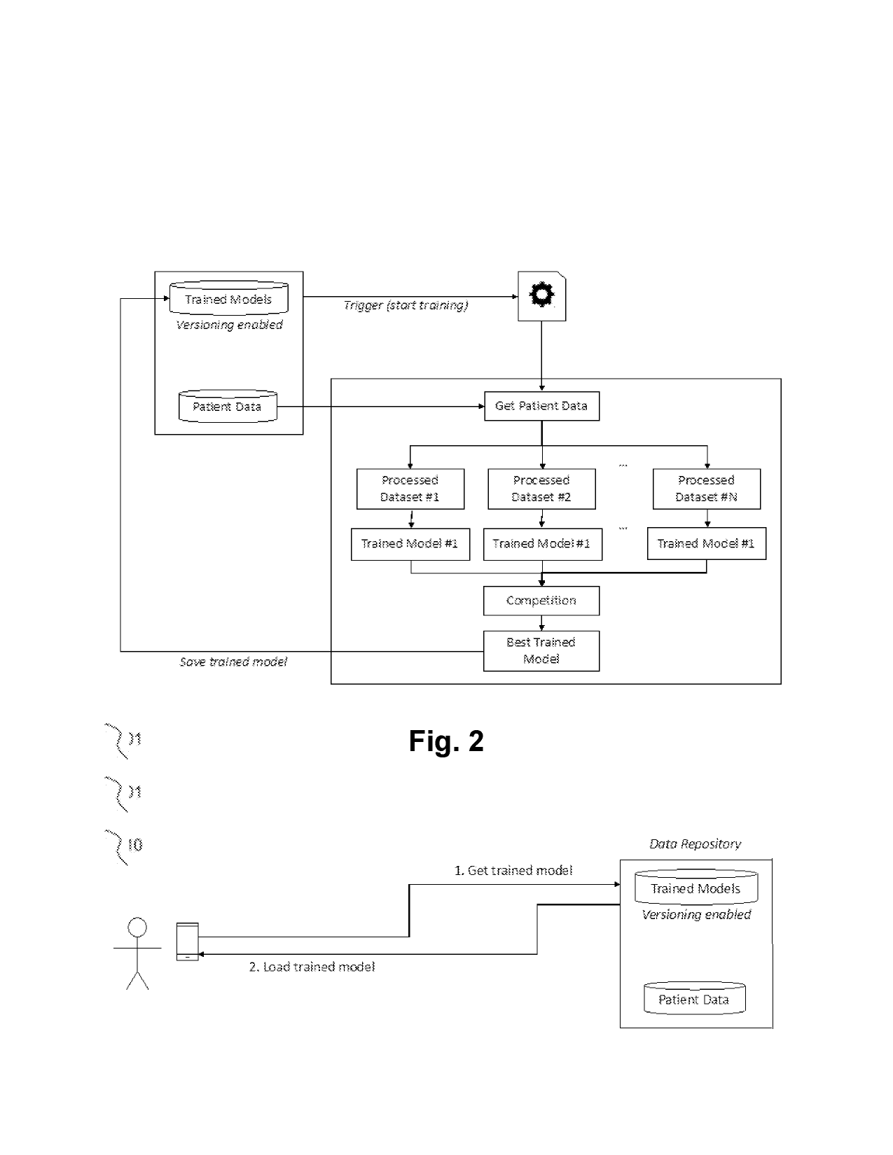

During the planning phase 101, the AI Engine 100 elaborates the behavioral model characterizing patients/users oxygen support needs vs the effort to be done. To this end, in the initial iterations, the data used to train the model (at least including heart rate and SPO2, and optionally geolocation data), comes from the set of similar patients/users, which are particularly separated into different groups according to the disease, gender, age, etc., along with the individual patient/user data obtained from the 6MWT. Then, in an embodiment, as shown in FIG. 2, the whole set of data can be split into N datasets through several artificial intelligence strategies, each training the data and generating a model according to several parameters. Finally, the N different models compete to check for accuracy, finally selecting the optimal one. The model selected is stored in a database 201 waiting to be queried during the runtime phase 102. This process can be handled using different artificial intelligence algorithms such as SVM, XGBOOST and/or Deep Learning. The rationale for using these algorithms is to balance different data training options, e.g., SVM is a supervised strategy suitable to classify data, XGBOOST supports large amount of data with multiple variables and Deep Learning is an unsupervised neural network strategy able to manage big data processes. The computational required by each artificial intelligence strategy is also different, hence the AI Engine 100 will also consider that variable to select the set of artificial intelligence strategies to be used, mainly when reaching out to very large amount of data.

As stated before, particularly, the system proposes to run the planning phase 101 at nights when no patient/user activity is reported (unless night activity is to be monitored as well). Indeed, the overall set of collected data is tentatively adjusted to 24 hours, thus putting together data enough to run a new training process, so each training iteration in the planning phase 101 will comprise data collected in one day.

In an embodiment, the planning phase 101 is run/executed in a cloud computing environment; not limitative as in other embodiments it can be run/executed by a computer device such as a mobile phone 10, among others.

### Runtime Phase

The runtime phase 102 starts when both the patient/user 1 wears the wearable device (to be monitored) and when the software APP is open. Basically, in this phase 102, the patient/user’s behavioral model computed in the planning phase 101 is queried with the real-time user collected data to obtain a prediction of the oxygen required by the patient/user 1 in the next time window.

In an embodiment, since queries to the behavioral model are triggered every minute, the prediction time window is also for 1 minute, meaning to say, that every minute the present invention computes and delivers an estimator of the real oxygen support need for next minute.

In a particular embodiment, the AI strategy to use in the runtime phase 102 is the one used to build the behavioral model delivered by the planning phase 101, i.e., the strategy computing the behavioral model selected after the competition process out of the N produced models during the planning phase 101. Alternatively, the AI strategy in this runtime phase 102 can be based in a recurrent neural network. In addition, as this is a real-time process, particularly all computation actions included in this phase 102 are done close to the patient/user 1, i.e., in a device physically deployed at the edge, either an ad-hoc system or the own patient/user’s mobile phone 10.

The Runtime phase 102 is split into two actions, start-up and prediction:

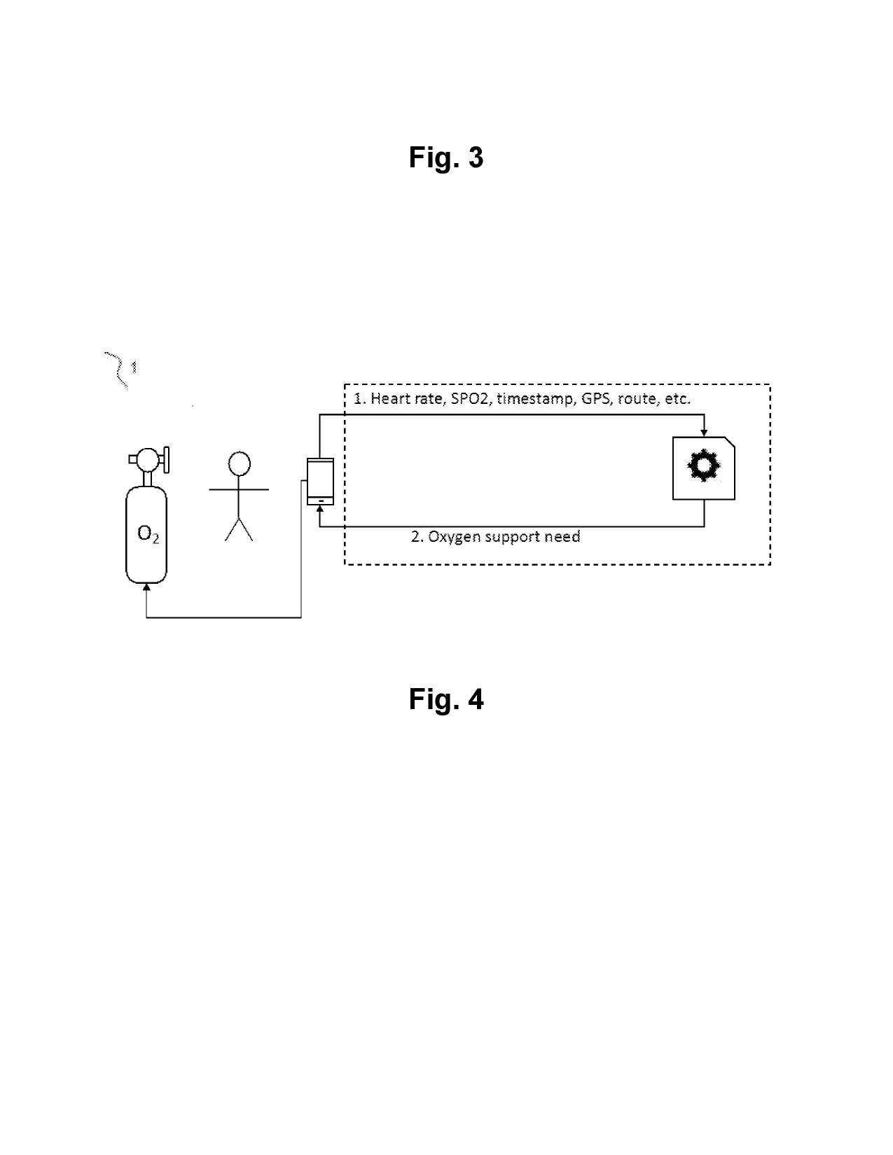

Start-up: This action is responsible for obtaining the last updated behavioral model trained with the last collected data (see FIG. 3). It consists on a request from the patient/user’s mobile phone 10 (or an ad-hoc device deployed as part of the present invention) to the database 201 where all trained behavioral models (i.e. the customized behavioral models) are stored. Consequently, the database 201 will return the last updated behavioral model. This action must guarantee that the patient/user’s mobile phone 10 (or an ad-hoc device) contains the last trained, or customized, behavioral model. Once the customized behavioral model is uploaded into the edge device and after some checks (queries to check response time, wrong values due for example to wrong collected data, etc.) next action starts. If checks are not successfully passed, an old version (day before) of the customized behavioral model will be used.

Prediction: This action is responsible for issuing the query to the behavioral model, including some values (e.g., Heart Rate, SPO2, and optionally GPS, Timestamp, route characteristics, etc.) and computing an estimator of the oxygen support need for the next time interval, e.g. next minute. This action will remain active while the software APP is on, see FIG. 4.

The whole set of data can be accessed through the two proposed frontends 104, 105, connected through the backend, also facilitating the direct communication between users, health professionals and caregivers.

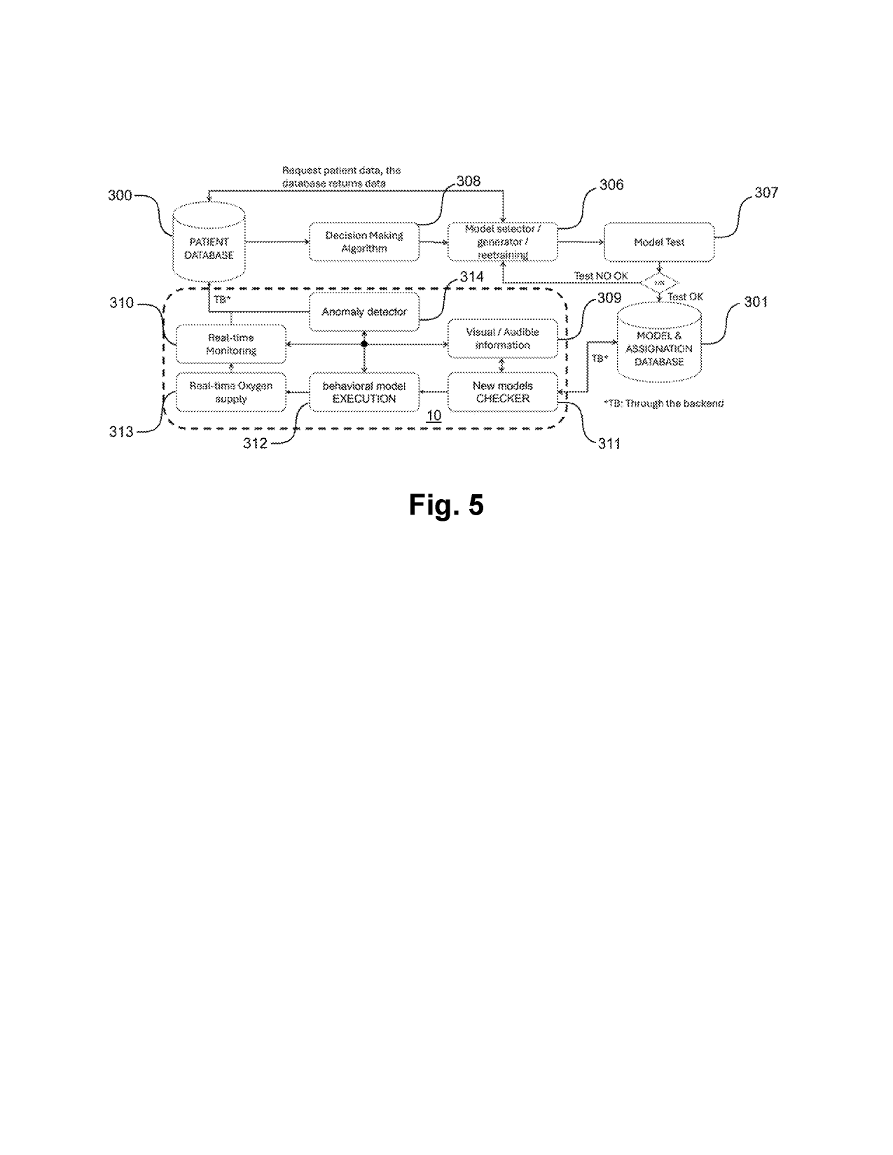

With reference now to FIG. 5, another embodiment of the proposed method is illustrated. In this embodiment, the method begins with real-time monitoring performed by a wearable data acquisition device (not shown). When a behavioral model of the patient/user is to be computed, an initial step comprises collecting monitoring data from the patient/user via the wearable data acquisition device. The monitoring data comprise oxygen saturation data. In other embodiments, additional data may also be collected, including pulse or heart rate (HR) data, geolocation data, oxygen concentrator flow rate data, predicted oxygen saturation (SpO₂) data, accelerometer data, wearable data acquisition device status information including battery status information, communication status information, etc. The collected monitoring data may subsequently be stored in a patient/user database 300.

Once the collected monitoring data has been stored, and following a decision made either by a patient/user, a healthcare professional, or a decision-making algorithm 308, the method proceeds 306 to one of three options: selecting a base behavioral model for the patient/user, generating a new behavioral model for the patient/user, or retraining an existing behavioral model associated with the patient/user. In the selection option, a decision-making algorithm 308 or a human operator selects a predefined generic or base behavioral model to be used for the patient/user.

According to an embodiment in which the selection option is determined by the decision-making algorithm, the decision may be categorized based on continuous data collection, data quality, a minimum required data set, prior experience regarding a minimum amount of patient data needed by a behavioral model generator to perform an action and satisfy a model test filter, differences in accuracy among previous predictions, and other criteria.

In the generation option, a behavioral model specific to the patient/user is computed/generated. For this purpose, a behavioral generator module requests, from the patient/user database 300, monitoring data deemed appropriate by the decision-making algorithm 308, the patient/user, or the generator module itself. Once generated, the behavioral model may be tested 307, and its validity may be evaluated based on prediction performance, including prediction success rates and error metrics.

In the retraining option, an existing behavioral model—such as a previously generated model, a base or generic model derived from different patients/users with similar pathologies, or a federated behavioral model—is trained using monitoring data of the patient/user captured up to that point, referred to as follow-up monitoring data. The generator module may request to the decision-making algorithm 308 that select from the patient/user database 300 the monitoring data considered relevant for retraining. Following retraining, the behavioral model may again be tested 307, and its validity is evaluated based on prediction performance. Retraining may further include behavioral model aggregation, the addition or modification of model layers, and the use of global, federated, or customized models.

In some embodiments, the data used for selecting, generating, or retraining behavioral models may additionally include synthetic data generated by artificial intelligence techniques, including generative artificial intelligence models and data generation methods.

Once a behavioral model is validated by a model testing process, the validated behavioral model is stored in a behavioral model and assignment database 301, which serves as a repository for behavioral models associated with different patients/users of the system.

After a behavioral model or its assignment has been configured in the behavioral model and assignment database 301, the user’s mobile device 10, through dedicated software, communicates with a backend system to determine whether a new behavioral model is available for the patient/user. If a new behavioral model is available, a new models checker module downloads the behavioral model and makes it available to the wearable data acquisition device.

Upon downloading the behavioral model, the user’s mobile device 10 provides a visual or audible notification indicating the availability of the new behavioral model. Through either manual or automatic interaction, which may be configurable, the patient/user executes the new behavioral model. The behavioral model then generates an oxygen delivery prediction 313 by processing data from a real-time monitoring cache 310.

The generated oxygen delivery prediction 313 is transmitted to a real-time oxygen supply module, which communicates, either wirelessly or via a wired connection, with an oxygen dosing device to indicate the amount of oxygen to be supplied at a given time. The real-time oxygen supply module may receive feedback data from the oxygen dosing device and communicates both the received data and confirmation of oxygen delivery to the real-time monitoring module 310.

The real-time monitoring module 310 communicates with one or more data collection devices and forwards the collected data to a behavioral model execution module 312 to generate updated oxygen predictions, to an anomaly detection module 314 to detect potential anomalies related to the patient/user or system components, and to a visual or audible information module 309 to provide the patient/user with status information regarding collected data and system operation.

If the anomaly detection module 309 detects an anomaly, it notifies the real-time monitoring module 310 to record the anomaly occurrence, the behavioral model execution module 312 if an adjustment to oxygen delivery is required, and the visual or audible information module 309 to inform the patient/user of the detected anomaly.

The visual or audible information module 309 can further communicate patient/user-initiated actions to other system modules, including notifying the anomaly detection module of patient/user-reported anomalies, instructing the behavioral model execution module 312 to execute newly downloaded behavioral models, prompting the new models checker module 311 to search for updated models, and instructing the real-time monitoring module 310 to initiate data capture when such capture is not configured to start automatically.

Both the real-time monitoring module 310 and the anomaly detection module 314 can communicate with the patient/user database 300 via the backend system 315 to store data collected by the various modules described above.

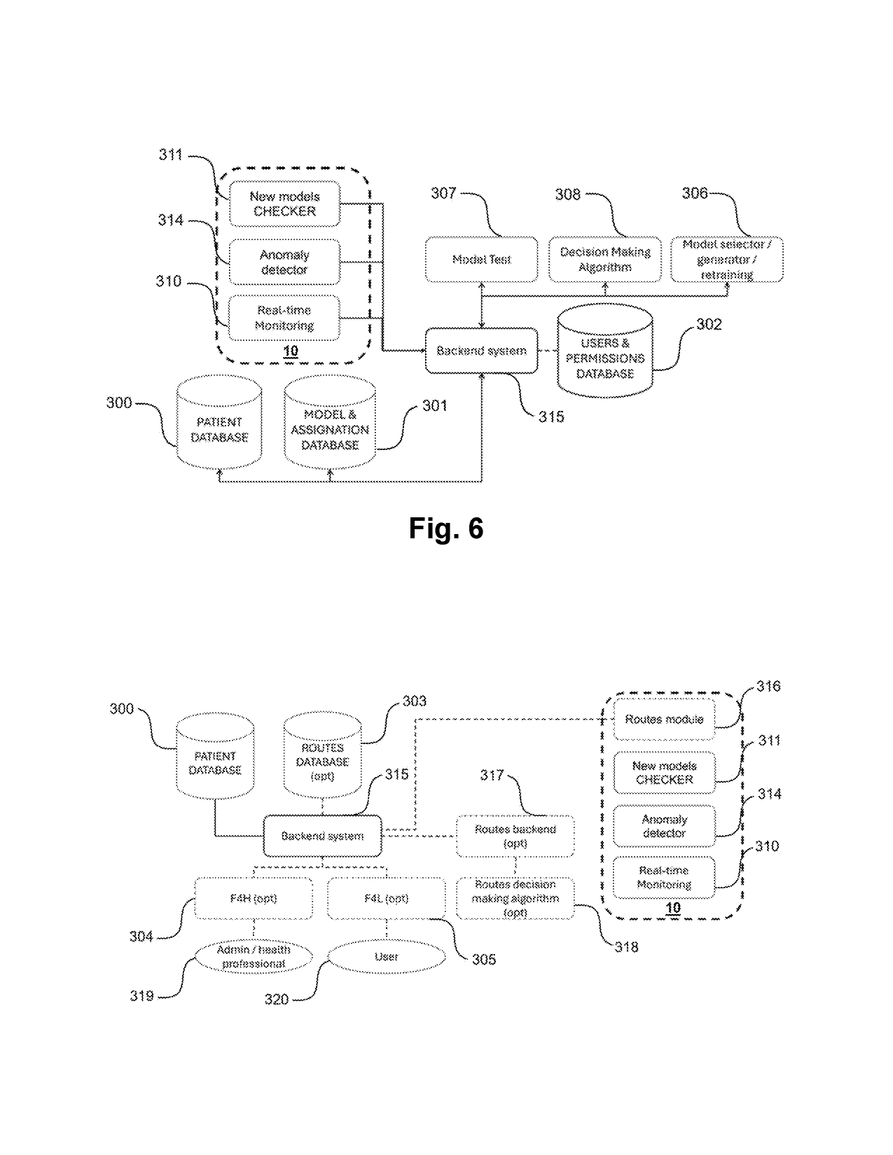

FIG. 6 shows an embodiment of the backend system 315, which is responsible for the correct management of the different elements/modules of the system. The “backend system” is accompanied by a database - independent of the system- for patients/users and permissions 302, that independently manages the viability of different patients/users to access one service or another, as well as the read, write, modification, and deletion access for the different tables or collections of the various databases. The figure also shows the connection between the user’s mobile device 10 with the backend 315. The user’s mobile device 10 requires patients/users to identify themselves and check permissions before performing any action in the system (they must previously validate against the “backend system” module to operate).

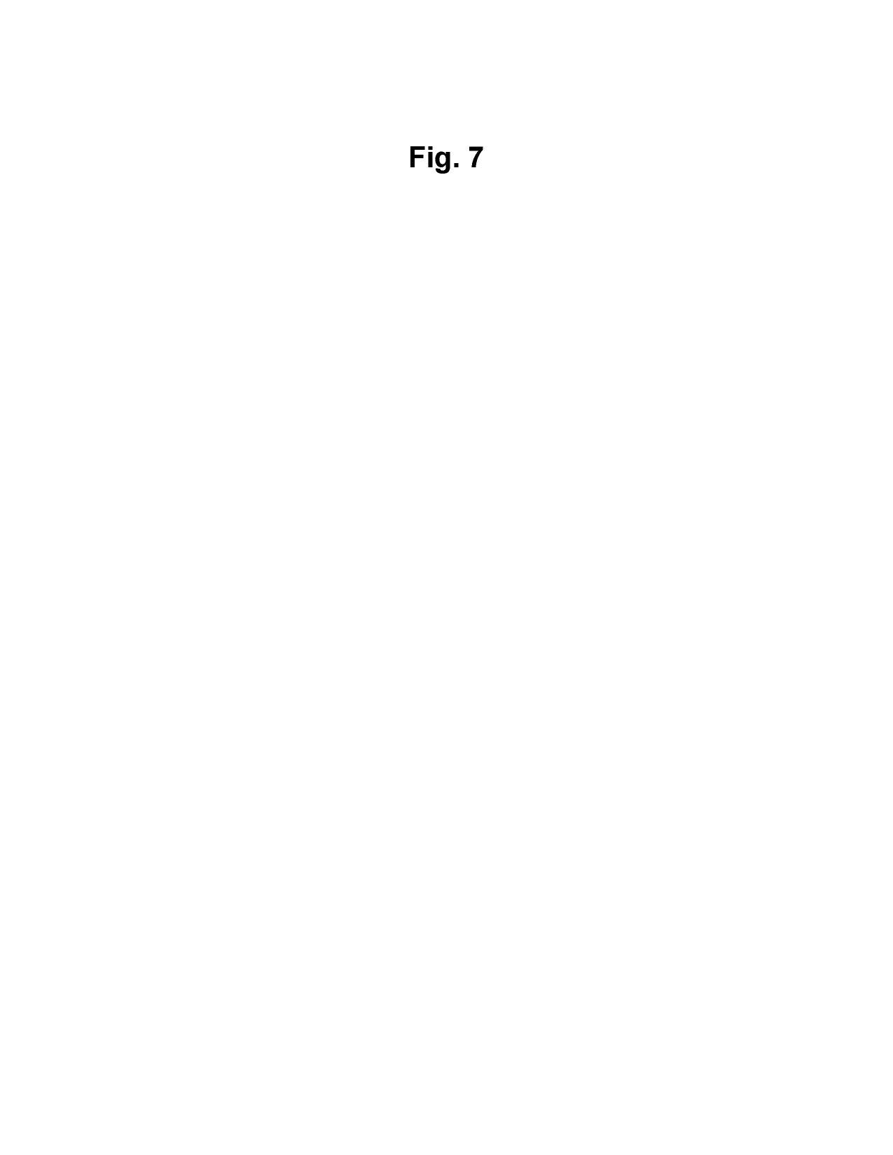

FIG. 7 illustrates another embodiment of dashboards and route management. The figure shows an example implementation in which frontends for healthcare professionals and patients/users, together with services for managing and creating stress test routes, are interconnected and architected. A routes database 303 is operatively connected to the backend system 315, thereby allowing the backend system 315 to make decisions based on route information and to use the routes database 303 across other system components, such as those illustrated in FIG. 6.

In the embodiment shown in FIG. 7, two types of visual frontends are provided for access by patients/users and healthcare professionals, namely F4H 304 and F4L 305. F4H (Frontend for Health) 304 is intended for medical professionals 319 and enables management of assigned patients/users, monitoring of results generated by the system, and review of detected anomalies. In some embodiments, F4H further enables the management, creation, and assignment of routes for patients/users.

F4L 305 is intended for end users 320 and enables them to view summaries and reports generated by the system, maintain communication with assigned healthcare professionals, review historical data collected by the system, and perform essential user management tasks. Access to and processing of user data through F4L 305 are configured to comply with applicable data protection regulations, including the European Union General Data Protection Regulation (GDPR).

In some embodiments, the system further comprises a routes service configured to define, manage, and assign routes that patients/users may follow to perform continuous physical activity while being remotely monitored by healthcare professionals, thereby reducing or eliminating the need for in-person visits to a medical center. The routes service may be configured through one or more frontends or through a routes decision-making algorithm module 318.

In such embodiments, the user’s mobile device 10 comprises a routes module 316 configured to coordinate with other modules of the patient/user device to manage assigned routes, guide route execution, and capture monitoring data associated with route performance.

The present invention can consider different potential incremental deployment options, as will be detailed next. However, beyond the conditions defined on each scenario, new approaches can be defined depending on the potential links and agreements to be defined with oxygen providers and pulse oximeters manufacturers.

In an embodiment, the software on the user’s mobile device 10 is deployed as an APP. The APP comprises two clear physical wireless interfaces, one to the wearable data acquisition device (e.g., a pulse oximeter) responsible for collecting the monitoring data from the patient/user 1 and the other one to an oxygen supply device or oxygen concentrator. The APP will collect the data, process it according to the algorithmic strategy already defined and will forward the decision on the oxygen to be delivered to the oxygen supply device, e.g., a portable oxygen concentrator. Although this is the lightest version, it includes all functionalities as envisioned for the present invention.

In an embodiment, the cited APP is extended, termed FOOXY suite or FOOXY O2, by adding specific hardware, thus having the need to use the user’s mobile device 10. The FOOXY suite interfaces the pulse oximeter 11 (see FIG. 4) through a wireless connection and connects the valve to a wired connection. Actually, the FOOXY Suite hardware may be deployed along with the cylinder. The complete FOOXY suite can include the electronic and the valve required to handle the oxygen dose. The present invention only requires a wireless connection to the pulse oximeter 11.

In some embodiments, the wearable data acquisition device (not shown in the figures) is a light bracelet responsible for data monitoring and collection. Moreover, the invention includes a box to be attached to the oxygen cylinder valve, responsible for the data processing and connectivity.

According to a particular embodiment, the present invention builds upon a database located at cloud premises storing all information collected from patients/users (e.g. the patient database), particularly under severe anonymized guarantees, to feed and train the AI engine 100 (or the behavioral model generator). When a new patient/user starts using the invention the method starts. The method can start by collecting some personal patient/user information, including age, disease, gender. This information is used to register the patient/user, thus facilitating the patient/user access to the F4L dashboard 105, 305, where the patient/user can visualize all data and statistics. Once done, the patient/user runs the 6MWT (or any other effort test or physical ctivity) and the invention starts collecting monitoring data. The present invention delivers the first time, the following outcomes:
A basal oxygen delivery dose, that can be considered by the health professional in a disease diagnose.
A behavioral model characterizing the patient/user oxygen needs vs effort done. This behavioral model is continuously (e.g. daily, weekly, etc.) trained with updated acquired activity data of the patient/user, so its inaccuracy lowers as time goes on.
Additionally, an exercise pattern defined as a set of routes (from the routes database) can be also proposed to the patient/user by the health professional, so preventing morbidity problems.

Then, every time the patient/user uses the present invention, the proposed system will monitor its behavior by at least collecting saturation, and optionally, heartbeat, localization parameters, HR data, oxygen concentrator flow rate data, etc. and will store the collected data in the patients/users database 108, 300 to train the behavioral model and recommend the oxygen support value when queried by the software APP. A warning signal can be provided to the patient/user and/or to the health professionals or caregivers if any potential issue happens during the runtime monitoring.

A check process can be included to guarantee that the AI Engine 100 is performing as expected. To that end, the estimated amount of oxygen is updated when the difference between the real-time monitoring and the estimated value goes far beyond a threshold, to be defined by the health professional. While this strategy guarantees that higher deviations (error in the estimator) are corrected, it also makes not necessary for the system to be continuously monitoring the status of the patient/user. In an embodiment, the checking policy advises for doing this process at the very beginning when the data used to train the AI is not individual, thus expecting higher deviations. However, as the data is updated with patient’s individual data, such checking can be shifted in time.

In an embodiment, the patient/user can look at the F4L information about his/her status, obtain statistics, and also use it to interact with other users. Similarly, the health professionals may use the F4P to see users’ status and also to get any warning and message coming from the patients/users. This data processing will be very useful to accommodate further treatments and to analyze users’ evolution and reaction to current efforts.

The artificial intelligence algorithms implemented in the present invention to train the AI engine or the behavioral model generator are adaptive, supporting the continuous incorporation of novel AI solutions. In certain embodiments, one or more neural networks are employed. In alternative embodiments, the artificial intelligence algorithms may include supervised, unsupervised, and reinforcement learning algorithms, evolutionary and optimization methods, and logic-based or knowledge-based systems.

According to the present invention, different activities can be also considered, including:
Tracking of the patient/user's activity: Includes predefined routes, exercise videos, feedback on the activity carried out, etc.
Activity diary: Journal containing the schedule of planned activities.
Communication: Deployment of communication in different environments, including doctor-user, user-user, user-caregiver, etc.
Educational: alimentation, prevention, good habits, medication intake control and reminders, general information on events, publications and studies, associations/foundation involvement.
Family support: The patient/user's relatives have a strong link with the disease and their environment despite not suffering the disease. The present invention must facilitate the connection and communication of the family as well as the customization of a fluid and simple communication and collaborative environment.
Alerts: Definition of the type of warnings to include in the system to facilitate both control by the medical team and self-control and monitoring by the patient/user himself and his environment.

The present invention can also give support and provide strategies to deal with some of the problems that the medical community has not yet solved, therefore including the following as basic functionalities:
Better management of physical activity as part of the non-pharmacological treatment, in the global strategy of patient management.
Customized and precise supply of individualized oxygen volume to each patient/user in response to real needs within the medical parameters defined by the medical chart.
Remote monitoring and real-time visualization of the patient/user's condition and its evolution based on the data obtained, the alerts displayed and the behavioral curves to be defined.
Objectivize the results obtained during routes or introducing manually or automatically exercise data like the 6MWT test to validate the degree and potential impact of the different hypoxemic diseases.
Share "best practices" through a collaborative medical platform to improve management strategies for these patients.
Allow a better and more accurate diagnosis on the potential impact of each disease on each patient/user specifically on the possible development of daily physical activities.
Facilitate the effort made to eliminate or reduce sedentary lifestyle, being considered a high risk factor in health for the entire population, through the generation that facilitates monitoring and adherence of patients/users to physical activity.
Interoperability, based on facilitating better coordination of medical personnel, by connecting patients/users, specialists and caregivers.
Data processing for the generation of data analytics that facilitate decision making for medical personnel, as well as any other derivatives that may be estimated in the future.

Various aspects of the proposed method, as described herein, may be embodied in programming. Program aspects of the technology may be thought of as "products" or "articles of manufacture" typically in the form of executable code and/or associated data that is carried on or embodied in a type of machine readable medium. Tangible non-transitory "storage" type media include any or all of the memory or other storage for the computers, processors, or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, from a management server or host computer of a scheduling system into the hardware platform(s) of a computing environment or other system implementing a computing environment or similar functionalities in connection with image processing. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to tangible “storage” media, terms such as computer or machine “readable medium” refer to any medium that participates in providing instructions to a processor for execution.

A machine-readable medium may take many forms, including but not limited to, a tangible storage medium, a carrier wave medium or physical transmission medium. Non-volatile storage media include, for example, optical or magnetic disks, such as any of the storage devices in any computer(s), or the like, which may be used to implement the system or any of its components shown in the drawings. Volatile storage media may include dynamic memory, such as a main memory of such a computer platform. Tangible transmission media may include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media may include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described herein may be embodied in a hardware device, it may also be implemented as a software only solution—e.g., an installation on an existing server. In addition, image processing as disclosed herein may be implemented as a firmware, firmware/software combination, firmware/hardware combination, or a hardware/firmware/software combination.

The present disclosure and/or some other examples have been described in the above. According to descriptions above, various alterations may be achieved. The topic of the present disclosure may be achieved in various forms and embodiments, and the present disclosure may be further used in a variety of application programs. All applications, modifications and alterations required to be protected in the claims may be within the protection scope of the present disclosure.

The scope of the present invention is defined in the following set of claims.

1. A method for determining an amount of oxygen to be delivered to a user having respiratory problems, the method comprising:
computing, by a processing unit, a behavioral model of the user, the behavioral model characterizing a relationship between oxygen saturation and physical effort of the user, wherein computing the behavioral model comprises training at least one artificial intelligence algorithm using monitoring data from the user, the monitoring data being collected by a wearable data acquisition device while the user performs at least one activity and comprising at least oxygen saturation data;
collecting, by the wearable data acquisition device, follow-up monitoring data from the user while the user performs an activity, the follow-up monitoring data comprising at least oxygen saturation data;
computing, by a processing unit, an estimated amount of oxygen to be delivered to the user based on the collected follow-up monitoring data and the behavioral model; and
adjusting, by a processing unit, the behavioral model using at least one artificial intelligence algorithm that utilizes the follow-up monitoring data.

2. The method of claim 16 wherein the monitoring data and the follow-up monitoring data further comprise one or more of pulse or heart rate (HR) data, geolocation data, oxygen concentrator flow rate data, predicted oxygen saturation (SpO₂) data, accelerometer data, wearable data acquisition device status information including battery status information and communication status information.

3. The method of claim 1, wherein collecting the monitoring data and follow-up monitoring data is performed at predetermined time intervals selected from the group consisting of daily intervals, two-day intervals, weekly intervals, and monthly intervals.

4. The method of claim 1, wherein computing the behavioral model further comprises using synthetic data generated by an artificial intelligence technique.

5. The method of claim 1, wherein adjusting the behavioral model further comprises using synthetic data generated by an artificial intelligence technique.

6. The method of claim 1, wherein the artificial intelligence algorithm comprises at least one neural network.

7. The method of claim 1, further comprising displaying at least one of the behavioral model and the computed estimated amount of oxygen on a screen of a computing device.

8. The method of claim 1, further comprising sending an audible or visual warning signal to the user and/or to a healthcare staff when the estimated amount of oxygen is different than a threshold.

9. The method of claim 1, wherein the activity comprises a physical activity or a daily activity of the user.

10. The method of claim 1, wherein the activity comprises the 6 minute walking test.

11. A method for determining an amount of oxygen to be delivered to a user having respiratory problems, the method comprising:
using, by a processing unit, a base behavioral model, the base behavioral model being selected from a set of previously computed behavioral models utilizing existing data from different patients/users, based on at least one of a disease type of the user, an effort capacity of the user, or an input provided by a healthcare staff member;
collecting, by the wearable data acquisition device, follow-up monitoring data from the user while the user performs an activity, the follow-up monitoring data comprising at least oxygen saturation data;
computing, by a processing unit, an estimated amount of oxygen to be delivered to the user based on the collected follow-up monitoring data and the base behavioral model; and
adjusting, by a processing unit, during a first iteration, the base behavioral model using at least one artificial intelligence algorithm that utilizes the follow-up monitoring data, generating an adjusted behavioral model as a result, and subsequently adjusting the adjusted behavioral model during successive iterations.

12. The method of claim 11, wherein the follow-up monitoring data further comprise one or more of pulse or heart rate (HR) data, geolocation data, oxygen concentrator flow rate data, predicted oxygen saturation (SpO₂) data, accelerometer data, wearable data acquisition device status information including battery status information, communication status information, and information relating to one or more nearby computing devices.

13. The method of claim 11, wherein collecting the follow-up monitoring data is performed at predetermined time intervals selected from the group consisting of daily intervals, two-day intervals, weekly intervals, and monthly intervals.

14. The method of claim 11, wherein adjusting the base behavioral model further comprises using synthetic data generated by an artificial intelligence technique.

15. The method of claim 11, wherein the artificial intelligence algorithm comprises at least one neural network.

16. The method of claim 11, further comprising sending an audible or visual warning signal to the user and/or to a healthcare staff when the estimated amount of oxygen is different than a threshold.

17. The method of claim 11, wherein the activity comprises a physical activity or a daily activity of the user.

18. The method of claim 11, wherein the activity comprises the 6 minute walking test.
